(19) United States
(12) Patent Application Publication
(10) Pub. No.: US 2006/0064135 A1
(43) Pub. Date: Mar. 23, 2006

(54) Implantable pressure sensor with pacing capability

(75) Inventor: Brian P. Brockway, Shoreview, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Transoma Medical, Inc., St. Paul, MN (US)

(21) Appl. No.: 11/223,587

(22) Filed: Sep. 8, 2005

Related U.S. Application Data
(63) Continuation-in-part of application No. 10/797,584, filed on Mar. 9, 2004.
(63) Continuation-in-part of application No. 10/077,566, filed on Feb. 15, 2002, now abandoned.
Application No. 10/077,566 is a continuation-in-part of application No. 09/968,644, filed on Oct. 1, 2001, now Pat. No. 6,947,795.
Application No. 09/968,644 is a continuation-in-part of application No. 09/825,130, filed on Apr. 3, 2001, now Pat. No. 6,659,959.
Application No. 10/077,566 is a continuation-in-part of application No. 09/491,233, filed on Jan. 25, 2000, now Pat. No. 6,379,308.
Application No. 09/491,233 is a continuation of application No. 09/264,147, filed on Mar. 5, 1999, now Pat. No. 6,296,615.
Application No. 09/264,147 is a continuation-in-part of application No. 09/159,653, filed on Sep. 24, 1998, now Pat. No. 6,409,674.
Application No. 09/159,653 is a continuation of application No. 08/950,315, filed on Oct. 14, 1997, now Pat. No. 6,033,366.
(60) Provisional application No. 60/608,077, filed on Sep. 8, 2004.
(60) Provisional application No. 60/454,951, filed on Mar. 12, 2003.
(60) Provisional application No. 60/269,922, filed on Feb. 19, 2001.

(51) Int. Cl. A61N 1/365

(52) U.S. Cl. 607/17

(57) ABSTRACT

Devices and methods for left ventricular or biventricular pacing plus left ventricular pressure measurement. For example, a pacing lead having a combined electrode and pressure sensor assembly may be used for left ventricular (LV) pacing and pressure measurement. The assembly may include one or more electrodes, a pressure sensor, and a pressure transmission catheter. Such a pacing lead is particularly suitable for biventricular pacing and may be incorporated into a cardiac resynchronization therapy (CRT) system, for example.

### CROSS-REFERENCE TO RELATED APPLICATIONS

The present application claims the benefit of U.S. Provisional Patent Application No. 60/608,077, filed Sep. 8, 2004, the entire disclosure of which is incorporated herein by reference. The present application is a continuation-in-part of U.S. patent application Ser. No. 10/077,566, filed Feb. 15, 2002, entitled Devices, Systems and Methods for Endocardial Pressure Measurement, and U.S. patent application Ser. No. 10/797,584, filed Mar. 9, 2004, entitled Devices and Methods for Detecting and Treating Inadequate Tissue Perfusion, the entire disclosures of which are incorporated herein by reference.

### BACKGROUND OF THE INVENTION

Congestive heart failure (CHF) is an end-stage chronic condition resulting from the heart's inability to pump sufficient blood, and is a significant factor in morbidity, mortality and health care expenditure. There are a variety of underlying conditions that may lead to CHF, and a variety of therapeutic approaches targeting such conditions. The selection of the therapeutic approach, and the parameters of the particular therapeutic approach selected, is a function of the underlying condition and the degree to which it affects the heart's ability to pump blood. Endocardial pressure, particularly left ventricular (LV) pressure, is a good indicator of the heart's ability to pump blood and the effectiveness of any given therapy.

Studies have shown that patients with moderate to severe CHF may benefit from cardiac resynchronization therapy (CRT). CRT devices are similar to conventional pacemakers, except that in addition to a lead for pacing the right ventricle, a CRT device includes a lead for pacing the left ventricle. Left ventricular leads may be placed intravascularly using a coronary sinus lead, or surgically using an epicardial lead. An example of a commercially available CRT device is the InSync® system from Medtronic. However, such CRT systems do not have the ability to measure LV pressure.

U.S. Pat. No. 5,353,800 to Pohndorf et al. describes a pacing lead that measures pressure using a hollow coiled needle. Pohndorf et al. describe measuring LV pressure by placing the lead in the right ventricular chamber with the coiled needle extending through the septal wall into the left ventricular chamber. Although Pohndorf et al. describe a lead for measuring LV pressure, Pohndorf et al. do not describe a lead for pacing the left ventricle as would be needed for a CRT system. Consequently, there is a need for a device and system capable of both LV pacing and LV pressure measurement.

### SUMMARY OF THE INVENTION

To address this need, the present invention provides devices and methods for left ventricular or biventricular pacing plus left ventricular pressure measurement. By way of example, not limitation, the present invention provides a pacing lead having a combined electrode and pressure sensor assembly for left ventricular pacing and pressure measurement. The assembly may include one or more electrodes, a pressure sensor, and a pressure transmission catheter. The assembly may be configured to be secured to the epicardial surface of the heart, and the pressure transmission catheter may be configured to extend through the heart wall. For example, the assembly may be positioned with respect to the heart such that the electrode is in a position to pace the LV, the pressure transmission catheter passes through a wall of the heart into the LV, and the pressure sensor resides outside the LV. Such a lead with a combined electrode and pressure sensor assembly for LV pacing and pressure measurement is particularly suitable for biventricular pacing and may be incorporated into a cardiac resynchronization therapy (CRT) system, for example. The measured LV pressure may be used in an open loop system providing LV pressure data to a physician, a closed loop system providing feedback control to a CRT system, or both, for example.

### BRIEF DESCRIPTION OF THE DRAWINGS

FIG. 1 is a schematic illustration of a pacing system including a combined pacing and pressure sensing lead for the left ventricle;

FIGS. 2A-2C are schematic illustrations of various electrode arrangements for the combined pacing and pressure sensing lead shown in FIG. 1;

FIG. 3 is a more detailed schematic diagram illustrating a combined pacing and pressure sensing lead;

FIG. 4 is a longitudinal cross-section of an alternative pressure transmission catheter; and

FIG. 5 is a longitudinal cross-section of an alternative pressure sensor arrangement.

FIG. 6 is a perspective view showing an illustrative assembly in accordance with an exemplary embodiment of the present invention.

FIG. 7 is a perspective view showing a pressure transmission catheter including a shaft.

FIG. 8 is an additional a perspective view showing the pressure transmission catheter shown in the previous figure.

FIG. 9 is a schematic illustration showing a body and a cardiac pacing system.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

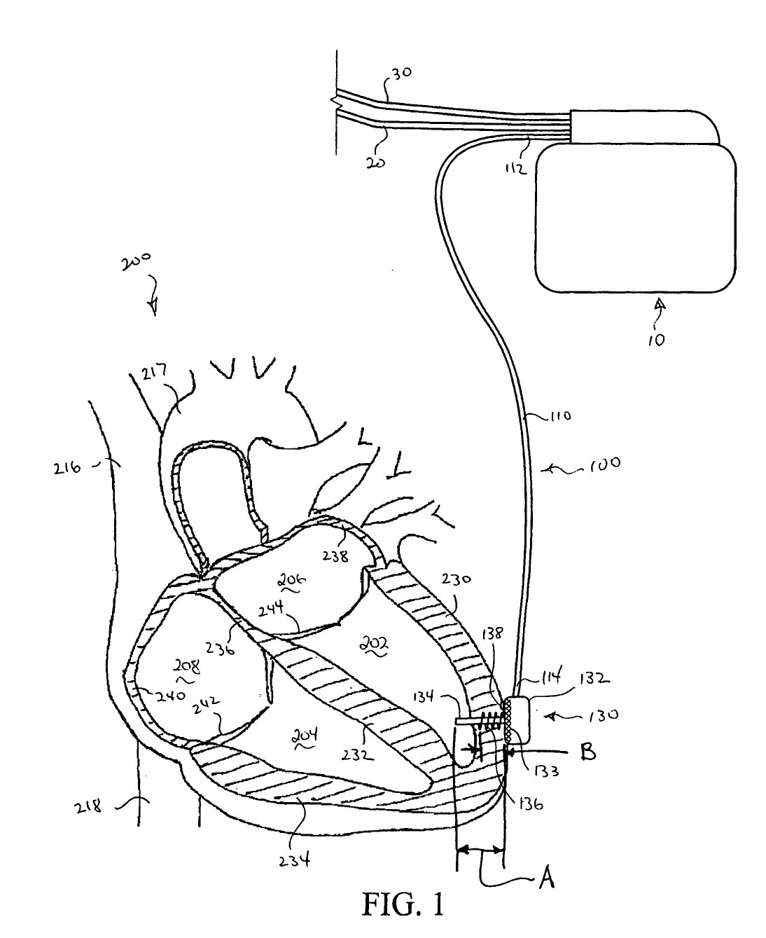

With reference to FIG. 1, a system for left ventricular pacing and pressure measurement is shown schematically. To facilitate a discussion of the system, it is helpful to define and label some of the anatomical features of the heart 200 shown in FIG. 1. The heart 200 includes four chambers, including the left ventricle (LV) 202, the right ventricle (RV) 204, the left atrium (LA) 206, and the right atrium (RA) 208. The LV 202 is defined in part by LV free wall 230, and the RV 204 is defined in part by RV free wall 234. The LV 202 and the RV 204 are separated by ventricular septal wall 232, and the LA 206 and the RA 208 are separated by atrial septal wall 236.

The right atrium 208 receives oxygen deprived blood returning from the venous vasculature through the superior vena cava 216 and inferior vena cava 218. The right atrium 208 pumps blood into the right ventricle 204 through tricuspid valve 242. The right ventricle 204 pumps blood through the pulmonary valve and into the pulmonary artery which carries the blood to the lungs. After receiving oxygen in the lungs, the blood is returned to the left atrium 206 through the pulmonary veins. The left atrium 206 pumps oxygenated blood through the mitral valve 244 and into the left ventricle 202. The oxygenated blood in the left ventricle 202 is then pumped through the aortic valve, into the aorta 217, and throughout the body via the arterial vasculature.

Returning to a discussion of the system illustrated in FIG. 1, the system generally includes a pulse generator 10 and a combined left ventricular (LV) pacing and pressure sensing lead 100. The pulse generator 10 may comprise a cardiac resynchronization therapy (CRT) device for biventricular pacing, or a combined CRT and defibrillation (CRT-D) device for biventricular pacing and defibrillation. Accordingly, the pulse generator 10 may accommodate three or four leads, for example, including an atrial sensing lead 20, a right ventricular (RV) therapy lead 30, and a LV pacing lead 100.

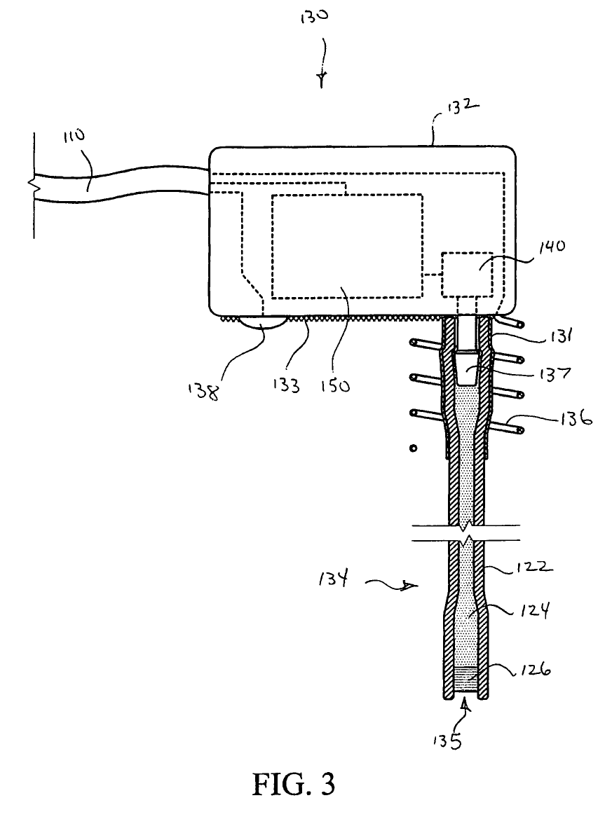

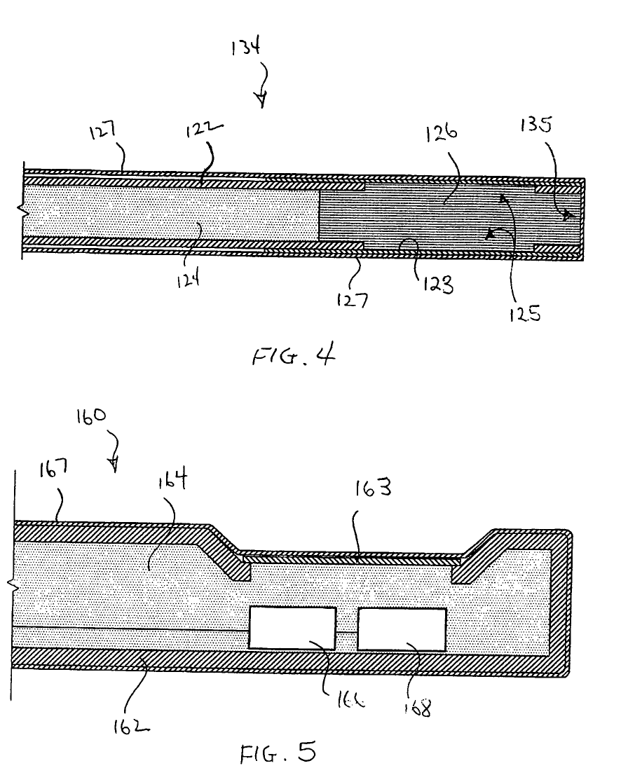

The LV lead 100 includes a lead body 110 having a proximal end portion 112 connected to the pulse generator 10 and a distal end portion 114 connected to an electrode and pressure sensor assembly 130. The electrode and pressure sensor assembly 130 may include a hermetically sealed housing 132 containing a pressure sensor and associated electronics as best seen in FIG. 3. A pressure transmission catheter (PTC) 134 may be connected to and extend from the housing 132, and may be configured to extend through a wall of the heart 200 and into a chamber, such as through the LV free wall 230 and into the LV chamber 202 as shown.

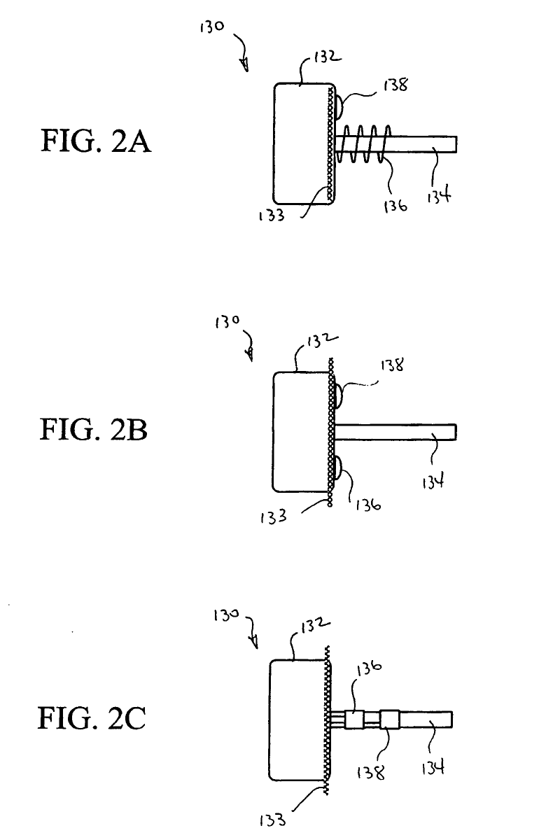

A pacing electrode 136 may be mounted to a portion of the assembly 130, such as around the PTC 134 as shown. A reference electrode 138 may be mounted to a portion of the assembly 130 and spaced from the pacing electrode 136, such as on the bottom side of the housing 132. Alternatively, a single electrode (e.g., 136 or 138) may be implemented by using control circuitry to periodically switch the function (e.g., pace or reference) of the electrode. In this alternative arrangement, the control circuitry (e.g., electronics module 150 as described hereinafter) would communicate the timing, pacing stimulus and sensing parameters to and from the electrode. As discussed in more detail with reference to FIGS. 2A-2C, the pacing electrode 136 and the reference electrode 138 may be mounted in a variety of places on the assembly 130 to effectively position the electrodes for pacing the LV via the myocardial and/or epicardial portions of the LV wall 230.

In FIG. 1 pacing electrode 136 is shown having a length B and PTC 134 is show having a length A. In some useful embodiments of the present invention, PTC 134 is configured to extend through the LV free wall 230 of the heart 200. In the embodiment of FIG. 1, for example, length A of PTC 134 is greater than the thickness of LV free wall 230. Also in the embodiment of FIG. 1, length B of pacing electrode 136 is smaller than the thickness of LV free wall 230. Some exemplary embodiments of the present invention include an electrode that is dimensioned to contact the outer wall of a left ventricle without contacting the blood disposed within the left ventricle. With reference to FIG. 1, it will be appreciated that distance A is greater than distance B.

With this arrangement, endocardial pressure (e.g., LV pressure) may be measured via the PTC 134, which refers blood pressure from within the chamber to the pressure sensor contained in the housing 132. The pressure sensor (or pressure transducer), together with the associated electronics in the housing 132, convert the pressure signal into an electrical signal (analog or digital) which is transmitted to the pulse generator 10 via lead body 110. Accordingly, lead body 110 may contain six (or more) conductors; one each for power, ground, control in, data out, pacing electrode 136, and reference electrode 138. Additional conductors may be provided in the lead body 110 to the extent that additional sensors (e.g., temperature sensor) or electrodes (e.g., ECG electrodes) are utilized. The electrical pressure signal received by the pulse generator 10 may be recorded, stored for later retrieval, or used to control pacing parameters or regimen. For example, the measured LV pressure may be used in an open loop system wherein telemetry is used to provide LV pressure data to a physician who can monitor the effectiveness of the therapy and modify the therapy as needed. Alternatively, the measured LV pressure may be used in a closed loop system wherein LV pressure data is used for feedback control of the pulse generator 10.

In some instances, it may be desirable to separate the combined electrode and pressure sensor assembly 130 into two parts; a sensor assembly portion and an electrode assembly portion. In this alternative embodiment, the electrodes may be separated from the sensor assembly and take the form of a conventional epicardial lead, and the sensor assembly may be essentially the same as before (less the electrodes). The sensor assembly portion and the epicardial lead portion may share a common lead connected to the pulse generator 10.

A tissue in-growth promoting surface 133 such as polyester fabric may be disposed on a bottom surface of the housing 132 to secure the assembly 130 to the epicardial surface of the heart 200, such as the epicardial surface of the LV free wall 230 as shown. In addition to the bottom surface of the housing 132, the tissue in-growth promoting surface 133 may also be disposed about the sides and top of the housing 132 to further enhance attachment to the outside of the heart wall. Other attachment means such as sutures, adhesive or the like may be used as in the alternative or in addition to the tissue in-growth promoting surface 133.

Reference may also be made to U.S. Pat. No. 4,846,191 to Brockway et al., U.S. Pat. No. 6,033,366 to Brockway et al., U.S. Pat. No. 6,296,615 to Brockway et al., and U.S. Published patent Application No. 2002/0120200 to Brockway et al. for examples of alternative embodiments of the sensor assembly 130 onto which the electrodes 136, 138 may be disposed.

The assembly 130 may be mounted to the LV free wall 230 by a conventional surgical technique, or a less invasive technique may be utilized, such as a transthoracic technique, where access to the cardiac space is gained via an intercostal approach or a subxyphoid approach as known in the art. Examples of suitable minimally invasive tools and methods are as described in U.S. Pat. No. 5,827,216 to Igo et al., assigned to Comedicus, Inc., and U.S. Pat. No. 4,972,847 to Dutcher et al., assigned to Enpath Medical, Inc., the entire disclosures of which are incorporated herein by reference. Examples of commercially available tools and related components include the PerDUCER® access device available from Comedicus, Inc, Columbia Heights, Minn., the MyoPore® sutureless unipolar epicardial pacing lead and the FasTac® myocardial lead implant tool manufactured by Enpath Medical, Minneapolis, Minn. Implantation of the system may take place during a contemporaneous open chest procedure (e.g., coronary artery bypass or valve repair/replacement), or the system may be implanted in a separate procedure.

As an example of a surgical technique, a surgeon may perform a median sternotomy, or mini-thoracotomy, cutting across the dermal layer, sub-dermal tissue layer, muscle layer, and sternum. The surgeon then cuts the pericardial sac to expose the heart 200, down to the LV apex. The PTC 134 is introduced through the LV free wall 230 and into the LV chamber 202 at the desired pacing location using a peelable-sheath introducer and a trocar. The trocar may be inserted into a lumen of the peelable sheath. The LV free wall 230 may be pierced with the trocar and the peelable sheath to form a hole in the LV free wall 2310. The trocar may be removed from the lumen of peelable sheath and the PTC 134 may be inserted into the lumen of the sheath. The peelable-sheath introducer facilitates insertion of the PTC 134 into the heart wall and protects the PTC 134 from damage that may otherwise occur during the insertion process. Following insertion of the PTC 134, the peelable-sheath introducer is removed by peeling it off the PTC 134 and around the assembly 130. A sheath retainer may be used to prevent splitting of the introducer inside the heart wall and to hold the assembly 130 in place while the introducer is removed.

After a subcutaneous pocket is created for the pulse generator 10, the lead body 110 may be tunneled from the cardiac space to the subcutaneous pocket and the proximal end 112 of the lead body 110 may be connected to the pulse generator 10. The other sensing, RV pacing, and defibrillation electrodes may be placed transvenously using conventional techniques, and subsequently connected to the pulse generator. The pocket and the chest are then closed.

As seen in FIG. 1, the combined electrode and sensor assembly 130 may be implanted on the heart 200 of a patient. In this exemplary embodiment, the PTC 134 is inserted directly into the left ventricle (LV) 202 across the left ventricular wall 230 for the purpose of measuring LV pressure. In particular, the housing 132 resides on the epicardial surface in the pericardial space, with the PTC 134 extending across the epicardium, myocardium and endocardium, and into the LV chamber 202. In this position, the pacing electrode 136 is in contact with the myocardium and the reference electrode 138 is in contact with the epicardium. This allows for pacing of the LV and for monitoring of pressure in the LV chamber 202 of the heart 200.

Although it is presently preferred to mount the assembly 130 on the LV free wall 230 in order to pace and measure pressure in the LV for biventricular pacing applications, for example, other implant positions are also possible. By way of example, not limitation, the assembly 130 may be implanted such that the distal end of the PTC 134 resides in any chamber of the heart 200, such as the LV 202, the RV 204, the LA 206, or the RA 208, to measure endocardial pressure in the respective chamber. Also by way of example, not limitation, the assembly 130 may be mounted such that the pacing electrode 136 and the reference electrode 138 contact any heart wall, such as the LV free wall 230, the RV free wall 234, the ventricular septum 232, the atrial septum 236, the LA free wall 238, or the RA free wall, to pace the respective chamber. These alternative mounting positions permit the combined pacing and pressure sensing lead 100 to be used to pace (or defibrillate) different hearts walls and measure pressure in different heart chambers.

With reference to FIGS. 2A-2C, various electrode arrangements are schematically illustrated by way of example, not limitation. The illustrated electrode arrangements may be used in whole or in part, and may be combined in a variety of different ways to provide many permutations of possible arrangements.

FIG. 2A shows, in more detail, the embodiment illustrated in FIG. 1, wherein the pacing electrode 136 comprises a metallic helical coil wound around the PTC 134, and the reference electrode 138 comprises a metallic button extending from the bottom of the housing 132. In this embodiment, the helical coil electrode is in contact with the myocardium, and the button electrode is in contact with the epicardium. The helical coil serves as both an electrode and as an anchor to secure the assembly 130 to the heart wall.

FIG. 2B shows the pacing electrode 136 and the reference electrode 138 comprising buttons extending from the bottom of the housing 132. In this embodiment, both button electrodes 136, 138 are in contact with the epicardium. A plurality of button electrodes distributed about the bottom surface of the housing 132 may be used (together with corresponding switching circuitry) to selectively switch between electrode pairs to obtain the desired pacing effect (e.g., to establish or maintain capture, to change thresholds, etc.).

FIG. 2C shows the pacing electrode 136 and the reference electrode 138 comprising metallic rings disposed around and spaced apart on the PTC 134. In this embodiment, both ring electrodes 136, 138 are in contact with the myocardium. A plurality of ring electrodes distributed along the length of the PTC 143 may be used (together with corresponding switching circuitry) to selectively switch between electrode pairs to obtain the desired pacing effect (e.g., to establish or maintain capture, to change thresholds, etc.). In one embodiment, one ring electrode may be used for pacing (i.e., active) and the other ring electrode may serve as a reference electrode.

With reference to FIG. 3, additional details of an example embodiment of the combined electrode and sensor assembly 130 are shown schematically. The assembly 130 includes a sensor 140 comprising a pressure transducer and an electronics module 150 contained within a housing 132. The assembly 130 further includes a pressure transmission catheter (PTC) 134 extending from the housing 132, a pacing electrode 136 extending around the PTC 134, and a reference electrode 138 disposed on the bottom of the housing 132.

The housing 132 protects the pressure transducer 140 and the electronics module 150 from the harsh environment of the human body. The housing 132 may be fabricated of a suitable biocompatible material such as titanium or ceramic and may be hermetically sealed. The proximal end of the housing 132 includes an electrical feedthrough to facilitate connection of the electronics module 150, the pacing electrode 136, and the reference electrode 138 to the flexible lead body 110. The distal bottom side of the housing 132 includes a pressure transducer header to facilitate mounting of the pressure transducer 140 and to facilitate connection to the PTC 134.

The pressure transducer 140 may be of the piezoresistive, optical, resonant structure, or capacitive type. For example, the pressure transducer may comprise a piezoresistive wheatstone bridge type silicon strain gauge available from Sensonor of Horton, Norway. Examples of suitable pressure transducers are disclosed in U.S. patent application Ser. No. 10/717,179, filed Nov. 17, 2003, entitled Implantable Pressure Sensors, the entire disclosure of which is incorporated herein by reference.

The electronics module 150 may provide excitation to the pressure transducer 140, amplify the pressure and EGM signals, and digitally code the pressure and EGM information for communication to the pulse generator 10 via the flexible lead body 110. The electronics module 150 may also provide for temperature compensation of the pressure transducer 140 and provide a calibrated pressure signal. A temperature measurement device may be included within the electronics module 150 to compensate the pressure signal from temperature variations. In an alternative embodiment, the electronics module 150 communicates or creates the stimulus to drive the pacing electrode 136.

The flexible lead body 110 connects the electronics module 150 of the assembly 130 to the pulse generator. The lead body 110 may contain, for example, six conductors; one each for power, ground, control in, data out, pacing electrode 136, and reference electrode 138. The lead body 110 may incorporate conventional lead design aspects as used in the field of pacing and implantable defibrillator leads. The lead body 110 may optionally include one or more EGM electrodes, and the number of conductors may be modified to accommodate the EGM electrodes.

The PTC 134, which is shown in longitudinal cross-section, may comprise a tubular shaft 122 with a liquid-filled (or gel-filled) lumen 124 extending therethrough to a distal opening or port 135 containing a barrier 126. The proximal end of the PTC 134 is connected to the pressure transducer 140 via a nipple tube 137 to establish a fluid path from the pressure transducer 140 to the distal end of the PTC 134. The PTC 134 thus refers pressure from the pressure measurement site to the pressure transducer 140 located inside the housing 132. The PTC 134 may optionally include one or more EGM electrodes or other physiological sensors as described in U.S. Pat. No. 6,296,615 to Brockway et al the disclosure of which is hereby incorporated by reference herein.

The barrier 126, which may comprise a gel plug and/or membrane, may be disposed in or over the distal opening 135 to isolate the liquid-filled lumen 124 of the PTC 134 from bodily fluids and to retain the fluid in the lumen, without impeding pressure transmission therethrough. In one embodiment, the fluid 124 is chosen to be a fluorinated silicone oil and the gel 126 is chosen to be dimethyl silicone gel. Further aspects of suitable fluids 124 and gels 126 are described in U.S. patent application Ser. No. 10/272,489, filed Oct. 15, 2002, entitled Improved Barriers and Methods for Pressure Measurement Catheters, the entire disclosure of which is incorporated herein by reference.

The PTC 134 may comprise a wide variety of materials, constructions and dimensions depending on the particular clinical application and the bodily tissue in which the PTC 134 resides when implanted. For example, the PTC 134 may comprise an extruded polycarbonate-polyurethane tube with a thermally formed proximal flare to accommodate the nipple tube 137, and a thermally formed distal flare to increase the area of the sensing surface and thereby reduce pressure measurement errors due to motion artifacts and thermal expansion artifacts. The PTC 134 may also incorporate a polyester fabric tube 131 or other surface modification. The PTC 134 may be annealed to improve its mechanical properties and may be etched in solvent or solvent vapors to remove frayed edges.

By way of example, not limitation, the PTC 134 may have an overall length of approximately 26 mm, a proximal flare length of approximately 6.0 mm, a distal flare length of approximately 5.5 mm, tapered transition lengths of approximately 2.0 mm, a mid-shaft inside diameter of approximately 0.025 inches, a proximal flare inside diameter of approximately 0.038 inches increasing to 0.059 inches to accommodate the nipple tube 137, a distal flare inside diameter of approximately 0.042 inches, and a wall thickness of approximately 0.015 inches, which are particularly suitable for LV pressure monitoring applications as shown and described with reference to FIG. 1. Various different lengths, diameters, tapers, flares, wall thicknesses, coatings, coverings, surface treatments, etc. may be incorporated into the PTC 134 depending on the application without departure from the present invention. Further details and alternative embodiments of the PTC 134 are described in U.S. patent application Ser. No. 10/799,931, filed Mar. 12, 2004, entitled Pressure Transmission Catheter for Implantable Pressure Sensors, the disclosure of which is incorporated herein by reference.

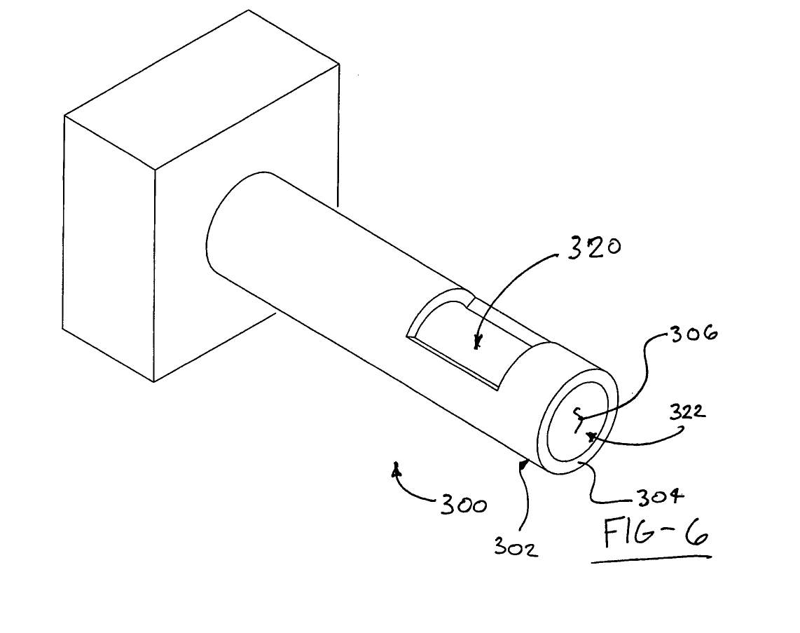

In some instances, it may be desirable to provide one or more side openings in the PTC 134 to increase the surface area for transfer of pressure into the fluid-filled lumen 124. An example of a side opening embodiment is illustrated in FIG. 4. In the illustrated embodiment, the PTC 134 includes a tubular shaft 122 having a distal opening 135 in addition to one or more side openings 125. The side openings 125 may be provided in addition to or in place of the distal port 135. If the distal port 135 is not used, it may be occluded with a suitable material such as epoxy or a polymer, for example. The side openings 125 may be any desired shape, such as circular ports or rectangular windows, for example.

The side ports 125 significantly increase surface area for pressure transmission. For example, a 1.0 mm inside diameter tubular shaft 122 has a distal port 135 area of 0.78 mm². The same sized tubular shaft 122 with two side windows each having a length of 3.00 mm and a height of 0.75 mm will add 4.50 mm² in opening area, an increase of 477%. Such side openings 125 provide several advantages, including increased sampling area and increased pressure transmission efficiency, especially in the event that the tip of the catheter becomes covered with fibrous tissue.

To retain the fluid 124 and the gel 126 inside the tubular shaft 122 of the PTC 134, a membrane 123 may be disposed over the side openings 125. In some useful embodiments of the present invention, membrane 123 comprises a resilient and/or reversibly deformable material. For example, membrane 123 may comprise an elastomeric material. The term elastomeric generally refers to a rubber-like material (e.g., a material which can experience about a 5% deformation and return to the undeformed configuration). Examples of elastomeric materials include rubber (e.g., natural rubber, silicone rubber, nitrile rubber, polysulfide rubber, etc.), thermoplastic elastomer (TPE), butyl, polyurethane, and neoprene. For example, the membrane 123 may comprise a thin walled (e.g., 0.002 inch thick wall) silicone rubber tube slid over the tubular shaft 122 adjacent the side openings 125. Silicone rubber that may be suitable in some applications is commercially available from Dow Corning Corporation of Midland, Mich. which identifies this silicone rubber using the SILASTIC trademark. Alternatively, the membrane 123 may comprise a thin walled polycarbonate-polyurethane that is bonded to the tubular shaft 122.

In addition to or in place of the thin membrane 123, a thin-walled cover 127 may be placed over all or a portion of the tubular shaft 122 (and membrane 123). The cover 127 may comprise a thin-walled tube or sock (closed-ended) that promotes tissue ingrowth (passivation) and that reduces the risk of thromboemboli formation. For example, the cover 127 may comprise a thin walled tube of ePTFE or a woven tube of Dacron.

In addition to the use of cover 127 over the tubular shaft 122 of the PTC 134, the use of a cover to reduce the risk of thromboemboli may also have significant benefit for a wide variety of other blood pressure sensor applications, particularly when the underlying material tends to promote the thromboemboli. For example, a covering may be useful for a blood pressure sensor 160 as shown in FIG. 5. The pressure sensor 160 schematically shown in FIG. 5 is similar to a pressure sensor described in U.S. Pat. No. 6,221,024 to Miesel, the entire disclosure of which is incorporated herein by reference.

The pressure sensor 160 includes a metallic housing 162 and a metallic diaphragm 163 defining an oil-filled cavity 164. A capacitive pressure transducer 166 and electronic integrated circuit 168 disposed in the cavity 164 detect changes in capacitance as a function of pressure impinging on the diaphragm 163. Because metallic materials in contact with blood flow in a vessel or chamber may tend to form thromboemboli, a thin cover 167 composed of a material such as ePTFE may be disposed about the housing 162 and/or the diaphragm 163 to reduce the likelihood therefor.

The pressure sensor 160 includes a housing 162 and a diaphragm 163 defining an fluid-filled cavity 164. In the exemplary embodiment of FIG. 5, housing 162 and diaphragm 163 both comprise metallic materials. In this exemplary embodiment, diaphragm 163 may be fixed to housing 162 by, for example, welding, brazing, and/or soldering. Examples of metallic materials that may be suitable in some applications include titanium, stainless steel, MP35N alloy, and platinum. A pressure transducer 166 and an electronic integrated circuit 168 disposed in the cavity 164 provide a signal S that changes as a function of pressure impinging on the diaphragm 163. In the embodiment of FIG. 5, a covering 167 is disposed over the housing 162 and diaphragm 163. Because metallic materials in contact with blood flow in a vessel or chamber may tend to form thromboemboli, a thin cover composed of a material such as, for example, ePTFE disposed about the housing 162 and/or the diaphragm 163 may reduce the likelihood that thromboemboli will form.

A number of materials may be suitable for use in covering 167. Examples of such materials include fluoropolytetrafluoroethylene (PTFE), ePTFE, polyethylene (PE), polypropylene (PP), polyvinylchloride (PVC), polyurethane, and DACRON. A number of manufacturing processes may be used to create covering 167. For example, covering 167 may be woven from a plurality of fibers. By way of a second example, covering 167 may be formed from one or more sections of shrink tubing. The shrink tubing sections may be positioned and then shrunk by the application of heat. A spray process may also be used to apply covering 167. For example, spraying PTFE solids in a suitable solvent carrier is a process which has been found suitable for this application. Another material that may be used to fabricate covering 167 is a thermoplastic generically known as parylene. There are a variety of polymers based on para-xylylene. These polymers are typically placed onto a substrate by vapor phase polymerization of the monomer. Parylene N coatings are produced by vaporization of a di(P-xylylene)dimer, pyrollization, and condensation of the vapor to produce a polymer that is maintained at comparatively lower temperature. In addition to parylene-N, parylene-C is derived from di(monochloro-P-xylylene) and parylene-D is derived from di(dichloro-P-xylylene). There are a variety of known ways to apply parylene to substrates. The use of paralene has been disclosed in U.S. Pat. No. 5,380,320 (to J. R. Morris), in U.S. Pat. No. 5,174,295 (to Christian et al.), and in U.S. Pat. No. 6,067,491 (to Taylor et al.). The entire disclosure of these United States Patents is hereby incorporated herein.

FIG. 6 is a perspective view showing an illustrative assembly 300 in accordance with an exemplary embodiment of the present invention. Assembly 300 comprises a shaft 302 having a wall 304 defining a lumen 306. In the embodiment of FIG. 6, wall 304 of shaft 302 defines a laterally oriented port 320 and an axially oriented port 322. With reference to FIG. 6 it will be appreciated that both laterally oriented port 320 and an axially oriented port 322 are disposed in fluid communication with lumen 306.

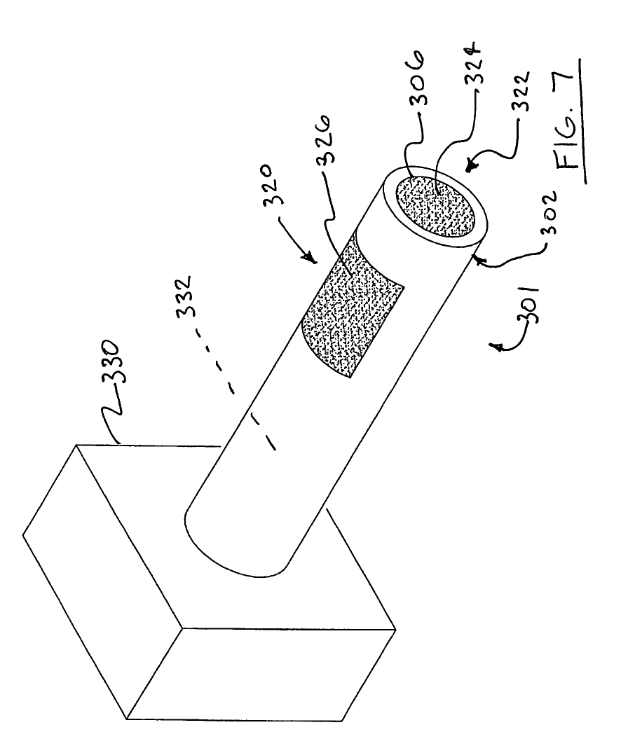

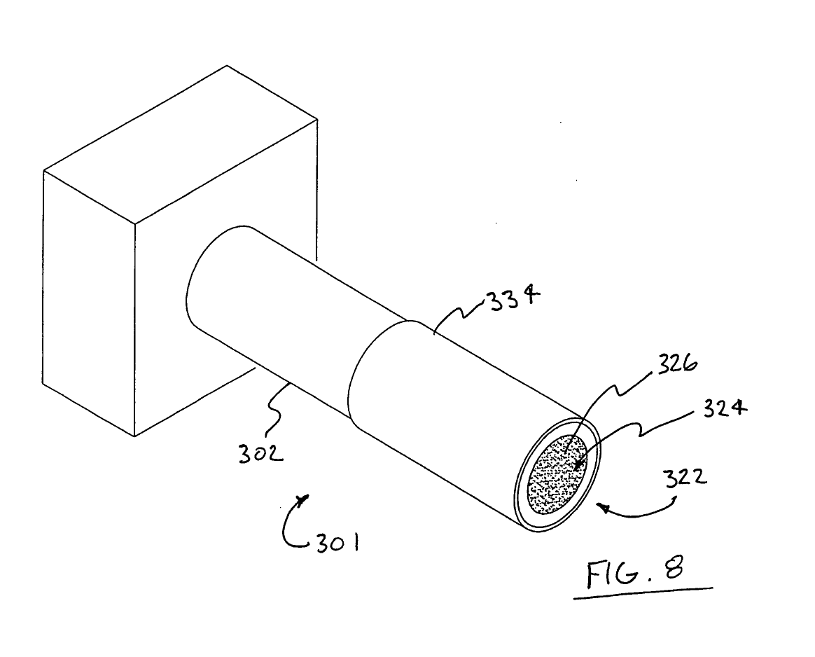

FIG. 7 is a perspective view showing a pressure transmission catheter 301 including shaft 302 shown in the previous figure. In the embodiment of FIG. 7, a gel plug 324 is disposed in lumen 306 proximate laterally oriented port 320 and axially oriented port 322. Also in the embodiment of FIG. 7, a pressure sensor 330 is disposed in fluid communication with lumen 306. A pressure transmitting fluid 332 is disposed in lumen 306 for transferring pressure between gel plug 324 and pressure sensor 330.

With reference to FIG. 7, it will be appreciated that a gel material 326 of gel plug 324 extends into laterally oriented port 320. In the embodiment of FIG. 7, an exposed surface area of gel material 326 extending into laterally oriented port 320 is generally equal to an outer surface area of laterally oriented port 320. Also in the embodiment of FIG. 7, an exposed surface area of gel material proximate axially oriented port 322 is generally equal to a lateral cross sectional area of lumen 306. In the exemplary embodiment of FIG. 7, the outer surface area of laterally oriented port 320 is greater than the lateral cross-sectional area of lumen 306.

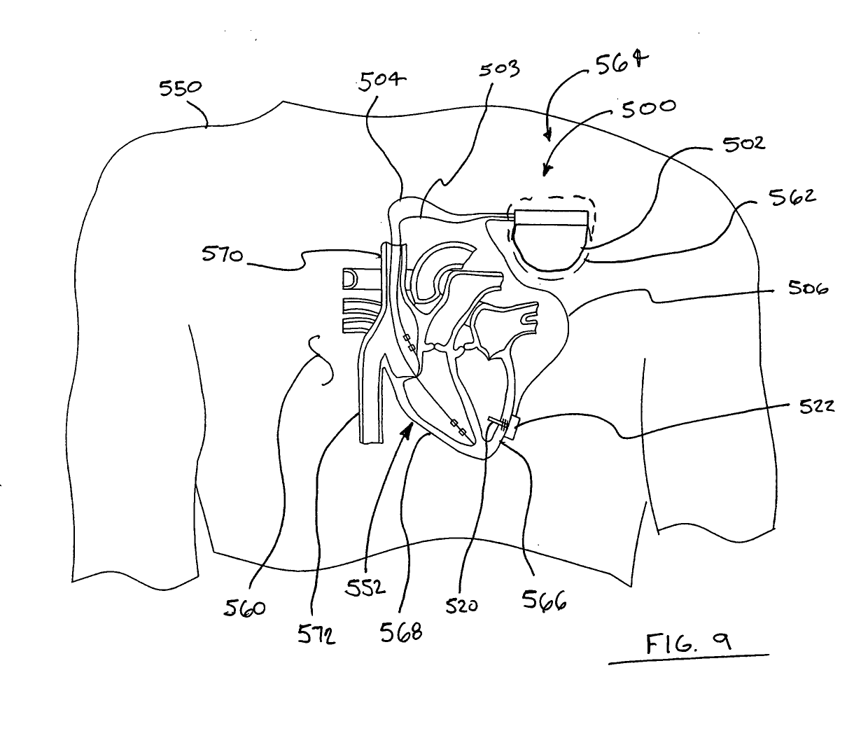

FIG. 8 is an additional a perspective view showing the pressure transmission catheter 301 shown in the previous figure. In FIG. 8, a membrane 334 is shown overlaying laterally oriented port 320. Also in FIG. 8, gel material 326 of gel plug 324 can be seen disposed in axially oriented port 322. Accordingly, it will be appreciated that membrane 334 covers laterally oriented port 320 and leaves axially oriented port 322 exposed in the exemplary embodiment of FIG. 8.

Membrane 334 may comprise various materials without deviating from the spirit and scope of the present invention. In some useful embodiments of the present invention, membrane 334 comprises a resilient and/or reversibly deformable material. For example, membrane 334 may comprise an elastomeric material. The term elastomeric generally refers to a rubber-like material (e.g., a material which can experience about a 5% deformation and return to the undeformed configuration). Examples of elastomeric materials include rubber (e.g., natural rubber, silicone rubber, nitrile rubber, polysulfide rubber, etc.), thermoplastic elastomer (TPE), butyl, polyurethane, and neoprene. Membrane 334 may comprise, for example, a thin walled (e.g., 0.002 inch thick wall) silicone rubber tube slid over shaft 302 adjacent laterally oriented port 320. Silicone rubber that may be suitable in some applications is commercially available from Dow Corning Corporation of Midland, Mich. which identifies this silicone rubber using the SILASTIC trademark. Alternatively, membrane 334 may comprise a thin walled polycarbonate-polyurethane that is bonded to shaft 302.

The material(s) and dimensions of membrane 334 may be selected such that membrane 334 transfers a pressure being measured to gel plug 324. At the same time, the materials and dimensions of shaft 302 may be selected to provide a pressure transmission catheter with a desired level of structural integrity. In some exemplary embodiments, shaft 302 may comprise a first material and membrane may comprise a second material different from the first material. For example, the second material may comprise an elastomeric material and the first material may comprise a non-elastomeric material. By way of a second example, the first material may have a first modulus of elasticity and the second material may have a second modulus of elasticity that is greater than the first modulus of elasticity. Shaft 302 may comprise various materials without deviating from the spirit and scope of the present invention. Examples of materials that may be suitable in some applications include polycarbonate, polyurethane (PU), polyethylene (PE), polypropylene (PP), and polyvinylchloride (PVC) fluoropolytetrafluoroethylene (PTFE), and ePTFE.

FIG. 9 is a schematic illustration showing a body 550 and a cardiac pacing system 500. Body 550 has a heart 552 that is disposed in a thoracic cavity 560 of body 550. With reference to FIG. 9, it will be appreciated that a pulse generator 502 of cardiac pacing system 500 is disposed in a pocket 562 formed in body 550. In the embodiment of FIG. 9, pocket 562 generally is disposed in a pectoral region 564 of body 550.

Heart 552 of body 550 includes a left ventricle 566 and a right ventricle 568. A plurality of blood vessels are shown connecting with heart 552 in FIG. 9. The blood vessels shown in FIG. 9 include a superior vena cava 570 and an inferior vena cava 572. In the embodiment of FIG. 9, cardiac pacing system 500 comprises a pulse generator 502, a right atrial lead 503, a right ventricular lead 504, and a left ventricular lead 506. The right ventricular lead 504 is connected to the pulse generator 502 and configured to pace the right ventricle 554 of the heart 552. In FIG. 9, right ventricular lead is shown passing through the superior vena cava 570 of body 550. The left ventricular lead 506 of cardiac pacing system 500 is connected to the pulse generator 502 and configured to pace the left ventricle 566 of the heart 552. In some useful embodiments of the present invention, the left ventricular lead 506 is also configured to measure left ventricular pressure. In the embodiment of FIG. 9, the left ventricular lead 506 comprises a pressure transmission catheter 520 and a housing 522. Housing 522 may contain a pressure sensor and associated electronics as shown, for example, in FIG. 3.

With reference to FIG. 9, it will be appreciated that left ventricular lead 506 extends between pulse generator 502 and the left ventricle 566 of the heart 552. With continuing reference to FIG. 9, it will be appreciated that a portion of left ventricular lead 506 is disposed within thoracic cavity 560 and that left ventricular lead 506 is outside of any blood vessels. Some methods in accordance with the present invention may comprise the step of positioning a conductor connected to an electrode and/or a pressure sensor so that it extends through a thoracic cavity without extending through any blood vessels.

From the foregoing, it will be apparent to those skilled in the art that the present invention provides, in exemplary non-limiting embodiments, devices and methods for left ventricular or biventricular pacing plus left ventricular pressure measurement, such as a pacing lead having a combined electrode and pressure sensor assembly for left ventricular pacing and pressure measurement. Further, those skilled in the art will recognize that the present invention may be manifested in a variety of forms other than the specific embodiments described and contemplated herein. Accordingly, departures in form and detail may be made without departing from the scope and spirit of the present invention as described in the appended claims. The entire disclosure of all patents and patent applications mentioned in this document are hereby incorporated by reference herein.

What is claimed is:

1. A method of treating a heart, comprising:
providing an electrode and sensor assembly including one or more electrodes, a pressure sensor and a pressure transmission catheter; and
positioning the electrode and sensor assembly proximate an outside surface of the heart such that the one or more electrodes reside on or in a wall of the heart, and the pressure transmission catheter is exposed to a heart chamber to provide fluid communication between the heart chamber and the pressure sensor.

2. A method as in claim 1, further comprising contacting the outside surface of the heart with the electrode.

3. A method as in claim 1, further including delivering the electrode and sensor assembly through an access tube extending into a pericardial space of the heart.

4. A method as in claim 3, further comprising delivering the electrode and sensor assembly via a transthoracic approach.

5. A method as in claim 1, further comprising delivering an electrical stimulus to the heart via the electrode.

6. A method as in claim 5, further comprising measuring endocardial pressure utilizing the pressure sensor and the pressure transmission catheter.

7. A method as in claim 6, wherein the electrical stimulus is delivered as a function of the measured endocardial pressure.

8. A method as in claim 7, wherein the electrical stimulus is delivered to a ventricle of the heart.

9. A method as in claim 1, further comprising contacting an epicardium of the heart with the electrode.

10. A method as in claim 1, further comprising contacting tissue under an epicardium of the heart with the electrode.

11. A method as in claim 1, further comprising piercing a wall of the heart with a trocar to form a hole.

12. A method as in claim 11, further comprising advancing a distal end of the pressure transmission catheter through the hole.

13. A method as in claim 12, wherein the pressure transmission catheter has a length that is greater than a thickness of the wall of the heart.

14. A method as in claim 1, further comprising piercing a wall of the heart with the electrode.

15. A method as in claim 1, further comprising advancing the electrode and sensor assembly through a pericardial space proximate the heart.

16. A method as in claim 1, further comprising advancing the electrode and sensor assembly into a thoracic cavity of a body.

17. A method as in claim 1, further comprising positioning a conductor to extend through the thoracic cavity of the body without extending through any blood vessel.

18. A method as in claim 1, further comprising positioning a conductor connected to the electrode and sensor assembly within the thoracic cavity and outside any blood vessels.

19. A method as in claim 1, further comprising the steps of:
connecting the conductor to a pulse generator; and
implanting the pulse generator in a pectoral region of a patient.

20. A method as in claim 19, further comprising positioning a conductor connected to the electrode and sensor assembly within a thoracic cavity of a body and outside the heart.

21. A method as in claim 1, further comprising:
providing a conductor connected to the electrode and sensor assembly;
providing a pulse generator; and
connecting the conductor to the pulse generator.

22. A method of treating a heart, comprising:
providing an electrode and sensor assembly including an electrode and a pressure sensor;
advancing the electrode and sensor assembly through a pericardial space proximate the heart; and
positioning the electrode and sensor assembly proximate an outside surface of the heart.

23. A method as in claim 22, further comprising delivering an electrical stimulus to the heart via the electrode.

24. A method as in claim 23, further comprising measuring endocardial pressure utilizing the pressure sensor.

25. A method as in claim 24, wherein the electrical stimulus is delivered as a function of the measured endocardial pressure.

26. A method as in claim 22, further comprising contacting an epicardium of the heart with the electrode.

27. A method as in claim 22, further comprising contacting tissue under the epicardium with the electrode.

28. A system for pacing a heart, comprising:
a left ventricular lead adapted to connect with a pulse generator the left ventricular lead being configured to pace a left ventricle of the heart; and
the left ventricular lead being configured to measure pressure in the left ventricular of the heart.

29. The system of claim 28, further comprising a pulse generator connected to the left ventricular lead.

30. The system of claim 29, further comprising a right ventricular lead connected to the pulse generator and configured to pace the right ventricle.

31. The system of claim 28, wherein the left ventricular lead comprises a distal portion comprising an electrode, a pressure sensor and a pressure transmission catheter.

32. The system of claim 31, wherein the pressure transmission catheter has a first length and the electrode has a second length that is different from the first length.

33. The system of claim 32, wherein the first length is greater than the second length.

34. The system of claim 33, wherein the first length is greater than a thickness of a wall of the heart and the second length is less than the thickness of the wall of the heart.

35. The system of claim 34, wherein the wall of the heart is an outer wall of the heart.

36. The system of claim 34, wherein the wall of the heart is a left ventricular free wall of the heart.

37. The system of claim 31, wherein the electrode is disposed about the pressure transmission catheter.

38. The system of claim 31, wherein the electrode is more rigid than the pressure transmission catheter.

39. The system of claim 31, wherein the electrode is sufficiently rigid to penetrate a wall of the heart.

40. The system of claim 31, wherein the electrode comprises a first material and the pressure transmission catheter comprises a second material different from the first material.

41. The system of claim 40, wherein the first material comprises an electrically conductive material and the second material comprises an electrically insulating material material.

42. The system of claim 40, wherein the first material comprises a metallic material and the second material comprises a polymeric material.

43. The system of claim 40, wherein the first material comprises a metallic material and the second material comprises a non-metallic material.

44. The system of claim 40, wherein the second material is more flexible than the first material.

45. The system of claim 40, wherein the first material has a first modulus of elasticity and the second material has a second modulus of elasticity that is smaller than the first modulus of elasticity.

46. A method of pacing a left ventricle of a patient's heart, comprising:
providing a pacing lead having a distal portion with one or more electrodes, a pressure sensor and a pressure transmission catheter;
positioning the pacing lead with respect to the heart such that the electrode is in a position to pace the left ventricle, the pressure transmission catheter passes through at least a portion of a wall of the heart into the left ventricle, and the pressure sensor resides outside the heart.

47. A method as in claim 46, wherein the position to pace the left ventricle is proximate a left ventricular free wall of the heart.

48. An apparatus, comprising:
a housing defining a cavity and an opening communicating with the cavity;
a diaphragm disposed over the cavity;
a pressure transducer disposed in the cavity;
a fluid disposed in the cavity for transferring pressure applied to the diaphragm to the pressure transducer; and
a covering disposed over the housing and the diaphragm.

49. The apparatus of claim 48, wherein the housing comprises a first material and the covering comprises a second material different from the first material.

50. The apparatus of claim 49, wherein the first material has a first thromboemboli forming characteristic and the second material has a second thromboemboli forming characteristic different from the first thromboemboli forming characteristic.

51. The apparatus of claim 49, wherein blood in contact with the first material is more likely to clot than blood in contact with the second material.

52. The apparatus of claim 48, wherein the housing and the diaphragm comprise metallic materials and the covering comprises a non-metallic material.

53. The apparatus of claim 48, wherein the housing and the diaphragm comprise metallic materials and the covering comprises a polymeric material.

54. The apparatus of claim 48, wherein the covering comprises a fabric.

55. The apparatus of claim 48, wherein the covering comprises a coating.

56. An apparatus, comprising:
a shaft having a wall defining a lumen and a laterally oriented port communicating with the lumen; and
a membrane extending over the laterally oriented port.

57. The apparatus of claim 56, further including a cover disposed over the membrane.

58. The apparatus of claim 57, wherein the cover comprises ePTFE.

59. The apparatus of claim 57, wherein the cover comprises a fabric.

60. The apparatus of claim 56, further including a gel plug disposed in the lumen proximate the laterally oriented port.

61. The apparatus of claim 60, further comprising a pressure sensor disposed in fluid communication with the lumen and a pressure transmitting fluid disposed in the lumen for transferring pressure between the gel plug and the pressure sensor.

62. The apparatus of claim 60, wherein a gel material of the gel plug extends into the laterally oriented port.

63. The apparatus of claim 56, further including an axially oriented port communicating with the lumen.

64. The apparatus of claim 63, wherein the membrane overlays the laterally oriented port and leaves the axially oriented port exposed.

65. The apparatus of claim 56, wherein the shaft comprises a first material and the membrane comprises a second material different from the first material.

66. The apparatus of claim 65, wherein the second material comprises an elastomeric material and the first material comprises a non-elastomeric material.

67. The apparatus of claim 65, wherein the second material is more flexible than the first material.

68. The apparatus of claim 65, wherein the first material has a first modulus of elasticity and the second material has a second modulus of elasticity that is smaller than the first modulus of elasticity.

69. The apparatus of claim 56, wherein the lumen has a first cross-sectional area and the laterally oriented port has a second cross-sectional area that is greater than the first cross-sectional area.
